(19) United States
(12) Patent Application Publication
(10) Pub. No.: US 2020/0197471 A1
(43) Pub. Date: Jun. 25, 2020

(54) S. SPINOSUM EXTRACT FOR TREATING FATTY LIVER DISEASE

(71) Applicant: ARIEL SCIENTIFIC INNOVATIONS LTD, Ariel (IL)

(72) Inventors: Tovit ROSENZWEIG, Kedumim (IL); Konstantin ROZENBERG, Rosh Haayin (IL)

(21) Appl. No.: 16/642,121

(22) PCT Filed: Aug. 28, 2018
(86) PCT No.: PCT/IL2018/050950

§ 371 Date: Feb. 26, 2020

Related U.S. Application Data
(60) Provisional application No. 62/552,538, filed on Aug. 31, 2017.

(51) Int. Cl. A61K 36/73; A61P 1/16; A61K 9/19; A61K 9/00; A23L 33/105

(52) U.S. Cl. CPC A61K 36/73; A61P 1/16; A23V 2002/00; A61K 9/0053; A23L 33/105; A61K 9/19

(57) ABSTRACT

The present invention provides Sarcopoterium spinosum (S. spinosum) extracts for use in preventing, treating and/or reducing the risk of developing fatty liver disease in a subject, compositions comprising the extracts, and methods for using them.

### FIELD OF INVENTION

The present invention relates to methods for treating fatty liver disease.

### BACKGROUND OF THE INVENTION

Fatty liver disease (FLD) is characterized by lipid accumulation in liver cells. The accumulated lipids cause cellular injury, sensitize the liver to further injuries and may also impair hepatic microvascular circulation. A number of factors may cause FLD including excessive alcohol (AFLD) and metabolic disorders, such as those associated with insulin resistance, obesity and hypertension. AFLD is highly prevalent and is one of the 20 leading causes of death worldwide. In the USA, the incidence may exceed 2 million cases. Non-alcoholic fatty liver disease (NAFLD) is also an extremely common condition, affecting up to ⅓ of the US population. A wide range of diseases and conditions can increase the risk of NAFLD, including: high cholesterol, high levels of triglycerides in the blood, obesity, polycystic ovary syndrome, sleep apnea, type 2 diabetes, hypothyroidism, and hypopituitarism, and cardiovascular disease. NAFLD is included in the metabolic syndrome which may be manifested by diabetes or pre-diabetes (insulin resistance), being overweight or obese, elevated blood lipids such as cholesterol and triglycerides, as well as high blood pressure.

AFLD and NAFLD have a similar pathogenesis and histology. The diseases cannot be distinguished at liver biopsy, and the differentiation between these two pathologies is based on ethanol intake (Bedogni et al., 2005).

FLD is characterized by excessive intrahepatic triglyceride content. Under the broad diagnosis of FLD is included a mild form, fatty liver, which manifests histologically by steatosis (the abnormal retention of lipids within a cell) alone.

Fatty liver may progress to a more severe form, steatohepatitis (non-alcoholic steatohepatitis (NASH) in the case of NAFLD), which is marked by the additional presence of lobar inflammation, hepatocellular ballooning and fibrosis. Liver fibrosis may lead to cirrhosis, which involves a risk for liver failure and hepatocellular carcinoma. Factors that may contribute to the development of steatohepatitis in both AFLD and NAFLD include: oxidative stress (imbalance between pro-oxidant and anti-oxidant chemicals that lead to liver cell damage); production and release of toxic inflammatory proteins (e.g. cytokines) by the patient's own inflammatory cells, liver cells, or fat cells; liver cell necrosis or death called apoptosis; adipose tissue (fat tissue) inflammation and infiltration by white blood cells; and gut microbiota (intestinal bacteria) which may play a role in liver inflammation. Factors that affect AFLD or NAFLD specifically include alcohol metabolism and insulin resistance, respectively. Both AFLD and NAFLD are characterized by alteration in hepatic lipid metabolism (Livero and Acco 2016).

However, while AFLD and NAFLD represent rather common causes of chronic liver disease, no specific therapies for these diseases, and their progressive form steatohepatitis, are currently available. Lifestyle modification is the cornerstone of management (restricting alcohol consumption and reducing body weight in AFLD and NAFLD, respectively). In addition, several medications are suggested for the treatment of the various clinical symptoms of the metabolic syndrome (associated with NAFLD), such as metformin for insulin resistance, telmisartan for hypertension and statins may be indicated for hyperlipidemia, and bariatric surgery is encouraged for high risk patients who do not improve with conservative management. However, no specific treatment is currently available for the treatment of this hepatic disorder. Similarly, there is no pharmacological strategy directed towards the treatment of AFLD. Thus, there is a great need for a new safe and effective therapy for both AFLD and NAFLD. It is projected that unless effective therapies for NAFLD are developed, NASH will become the leading indication for liver transplantation in the United States within 10-20 years.

Sarcopoterium spinosum (S. spinosum) is a chamaephyte of the Rosaceae family. Its branches are wooden, end in branched thorns and grow to a length of 30-40 cm. In the summer the green winter leaves at the end of the branches develop into thorns and are replaced by tiny leaves. S. spinosum has been used in folk medicine for its antidiabetic effect (Smirin et al., 2010, Journal of Ethnopharmacology 129(1):10-17; Rosenzweig et al., 2007, Israel Journal of Plant Sciences, 55(1):103-109). WO 2010/143140 discloses treatment or prevention of diabetes by administration of an extract from S. spinosum.

### SUMMARY OF THE INVENTION

In one aspect, the present invention provides a Sarcopoterium spinosum (S. spinosum) extract for use in preventing, treating and/or reducing the risk of developing fatty liver disease in a subject.

In a further aspect, the present invention provides a pharmaceutical composition comprising an extract of Sarcopoterium spinosum according to the invention, for use in preventing, treating and/or reducing the risk of developing fatty liver disease in a subject.

In a further aspect, the present invention provides a nutraceutical composition comprising an extract of Sarcopoterium spinosum according to the invention, for use in preventing, treating and/or reducing the risk of developing fatty liver disease in a subject.

In an additional aspect, the present invention provides a method for preventing, treating and/or reducing the risk of developing, fatty liver disease in a subject, comprising administering to said subject an extract of Sarcopoterium spinosum.

### BRIEF DESCRIPTION OF THE DRAWINGS

FIGS. 1A-1C show that preemptive treatment with S. spinosum improves glucose tolerance in high fat diet (HFD)-fed mice. C57BL/J mice were fed a standard diet (STD, circle) or HFD with (triangle in A or empty square in B) or without (full square) S. spinosum extract given as their drinking water (according to prevention protocol). (A) Body weight was measured every week. (B) Glucose tolerance test (GTT) was performed at age of 15 weeks as described in Materials and Methods. (C) Fasting serum insulin levels was measured at age of 17 weeks. The result are presented as mean±SE, *p<0.05, **p<0.005, ***p<0.0005 by student's t-test, compared to HFD-fed mice.

FIGS. 2A-2B show that preemptive treatment with S. spinosum enhances insulin signaling in liver of HFD-fed mice. C57BL/J mice model were fed STD, HFD or HFD+S. spinosum (according to prevention protocol), livers were removed at the age of 17 weeks. (A) Western blot analysis was performed on liver lysate using specific antibodies. These are representative results of three independent experiments. The bar graph in (B) is the result of optical density measurements of Western blots in A. Each bar represents the mean±SE of data obtained from 4 mice. Empty bar (left)—STD, gray bar (middle)—HFD, Black bar (right)—HFD+S. spinosum. *p<0.05 and **p<0.005 in Student's t-test. *p<0.05 and ***p<0.0005 in Student's t-test.

FIGS. 3A-3D show the effect of preemptive treatment with S. spinosum extract on hepatic glycogen and lipid content in HFD-fed mice. C57BL/6J mice were fed STD or HFD with or without S. spinosum extract given as their drinking water (according to prevention protocol). Mice were sacrificed at age of 17 weeks and hepatic glycogen (A), triglycerides (B) and total cholesterol (C) levels were measured. The results are presented as mean±SE (n>5 mice). *p<0.05, **p<0.005 by student's t-test, compared to HFD-fed mice. (D) H&E staining of livers of mice fed with STD or HFD and treated by S. spinosum extract. Arrows point on representative macro-steatosis (in HFD-fed mice) and micro-steatosis (in HFD+S. spinosum).

FIGS. 4A-4D show that preemptive treatment with S. spinosum extract increased liver mRNA expression of glucokinase (GCK). C57BL/J mice were fed STD, HFD or HFD+S. spinosum (according to prevention protocol), liver was removed at the age of 17 weeks. mRNA expression of (A) glucose-6 phosphatase (G6Pase), (B) phosphoenolpyruvate carboxykinase (PEPCK), (C) GCK, and (D) glucose transporter (GLUT)-2 was measured by real-time PCR. Results were normalized to the expression of the housekeeping gene, hypoxanthine-pyruvate Hypoxanthine-guanine phosphoribosyltransferase (HPRT). ***P<0.0005 by Student's t-test.

FIGS. 5A-5G show that preemptive treatment with S. spinosum extract reverses the alteration in mRNA expression of genes involved in lipid metabolism induced by HFD. C57BL/J mice were fed STD, HFD or HFD+S. spinosum (according to prevention protocol), liver was removed at the age of 17 weeks as described in methods. mRNA expression of the indicated genes (PPARα (A), PPARγ (B), ACC1 (C), SREBP-1c (D), SREBP2 (E), FAS (F), and HSL (G)) was measured by real-time PCR. Results were normalized to the expression of housekeeping gene, HPRT. ***P<0.0005 by Student's t-test.

FIGS. 6A-6B show that preemptive treatment with S. spinosum extract increases liver mRNA expression of AdipoR2. C57BL/J mice were fed STD, HFD or HFD+S. spinosum (according to prevention protocol), liver was removed at the age of 17 weeks. mRNA expression of adiponectin receptors AdipoRl (A) and AdipoR2 (B) was measured by real-time PCR. Results were normalized to the expression of housekeeping gene, HPRT. ***P<0.0005 by Student's t-test.

FIGS. 7A-7E show that treatment with S. spinosum improves glucose tolerance in high fat diet (HFD)-fed mice. C57BL/J mice were fed a standard diet (STD) or HFD with or without S. spinosum dried extract (according to treatment protocol). (A) Body weight was measured every week. (B) Glucose tolerance test (GTT) was performed at age of 15 weeks as described in Materials and Methods. (C and D) insulin tolerance test was performed at age of 16 weeks as described in Materials and Methods. The results are presented as absolute (C) or relative (D) values. (E) Fasting serum insulin levels was measured at age of 17 weeks. The result are presented as mean±SE, *p<0.05, **p<0.005, ***p<0.0005 by student's t-test, compared to HFD-fed mice. STD: filled circle; HFD: filled square; HFD+S. Spinosum extract (SSE) 30 mg/day: X; 60 mg/day: empty circle; 90 mg/day: empty diamond.

FIGS. 8A-8D show the effect of treatment with S. spinosum extract on hepatic lipid content in HFD-fed mice. C57BL/6J mice were fed STD or HFD with or without S. spinosum dried extract (according to treatment protocol). Mice were sacrificed at age of 17 weeks and hepatic triglycerides (A), and total cholesterol (B) levels were measured. The results are presented as mean±SE. *p<0.05, **p<0.005 by student's t-test, compared to HFD-fed mice. (C) Severity of NAFLD was evaluated by an independent pathologist as described in Materials and Methods. (D) H&E staining of livers of mice fed with STD or HFD and treated by SSE at the indicated doses. Arrows point to representative steatotic hepatocytes.

FIGS. 9A-9D show that treatment with S. spinosum did not affect glucose tolerance in western diet (WD)-fed mice. C57BL/J mice were fed a standard diet (STD) or WD with or without S. spinosum dried extract (according to treatment protocol). (A) Body weight was measured every week. (B) Glucose tolerance test (GTT) was performed at age of 18 weeks as described in Materials and Methods. (C) Insulin tolerance test was performed at age of 19 weeks as described in Materials and Methods. (D) Fasting serum insulin levels was measured at age of 20 weeks. The result are presented as mean±SE, *p<0.05, **p<0.005, ***p<0.0005 by student's t-test, compared to WD-fed mice. STD: filled circle; WD: filled square; WD+S. Spinosum extract (SSE) 30 mg/day: X; 60 mg/day: empty circle; 90 mg/day: empty diamond.

FIGS. 10A-10D show the effect of treatment with S. spinosum extract on hepatic lipid content in WD-fed mice. C57BL/6J mice were fed STD or WD with or without S. spinosum dried extract (according to treatment protocol). Mice were sacrificed at age of 20 weeks and hepatic triglycerides (A), and total cholesterol levels (B) were measured. The results are presented as mean±SE. *p<0.05, **p<0.005 by student's t-test, compared to WD-fed mice. (C) H&E staining of livers of mice fed with STD or WD and treated with the indicated doses of SSE. Arrows point to representative steatotic hepatocytes, circles mark foci of inflammation. (D) Severity of NAFLD was evaluated by an independent pathologist as described in Materials and Methods.

FIG. 11 shows that treatment with S. spinosum extract reduced serum ALT in WD-fed mice. C57BL/6J mice were fed STD or WD with or without S. spinosum dried extract (according to treatment protocol). Mice were sacrificed at age of 20 weeks and serum ALT was measured as described in Materials and Methods. The result are presented as mean±SE, *p<0.05 by student's t-test, compared to WD-fed mice.

### DETAILED DESCRIPTION OF THE INVENTION

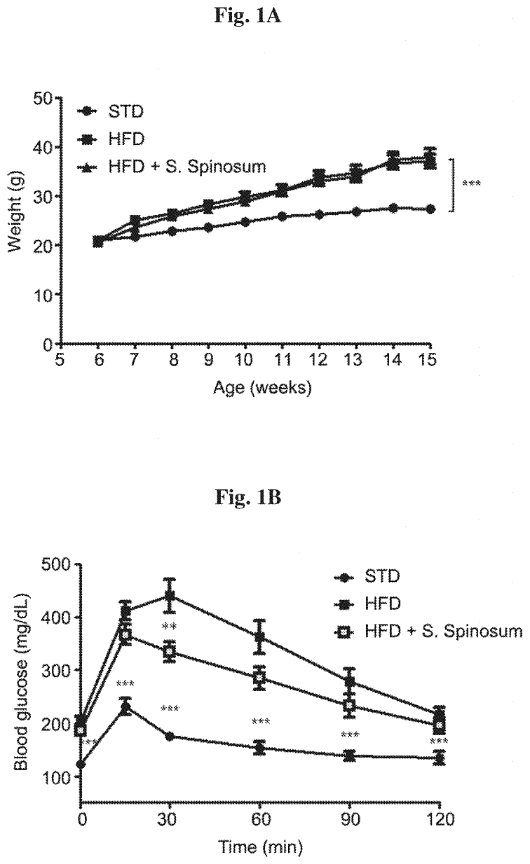

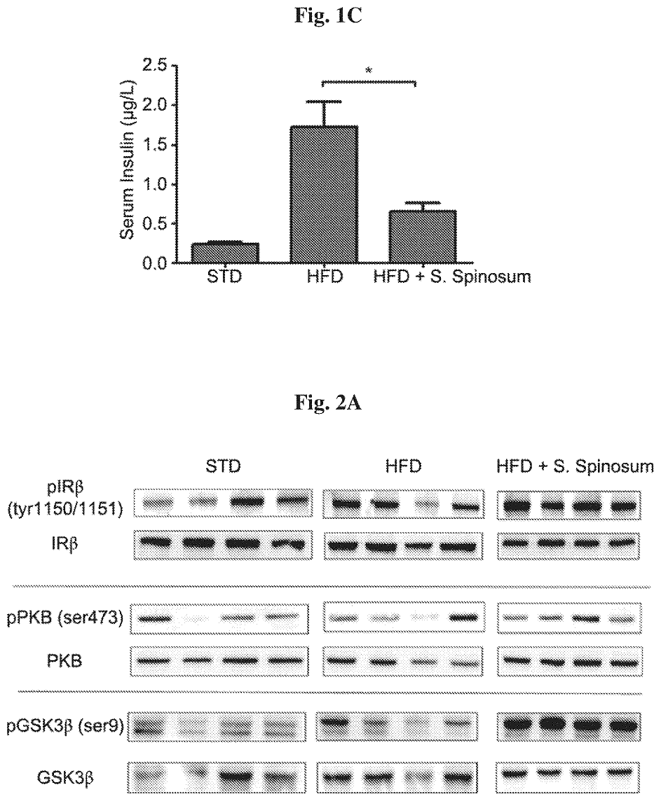

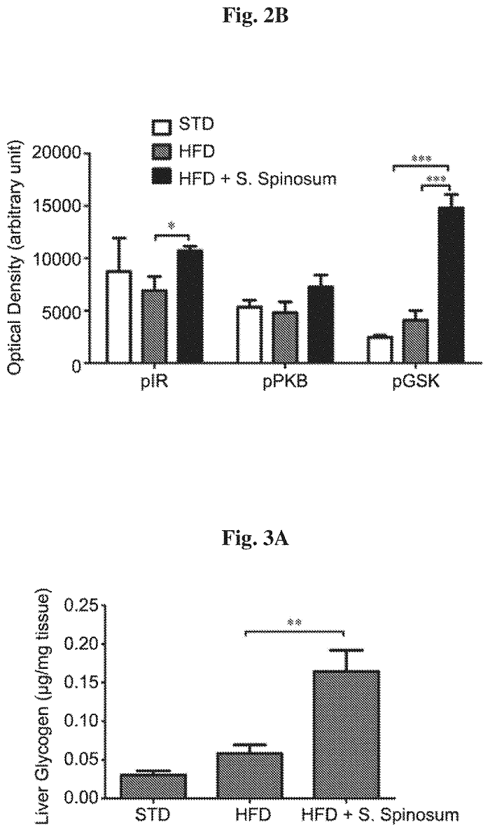

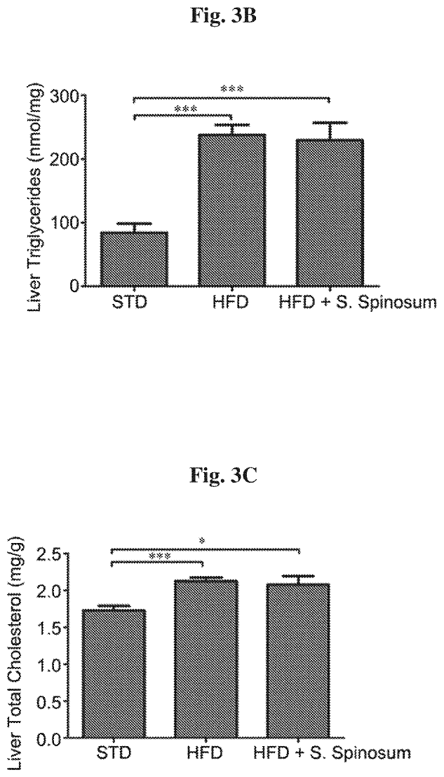

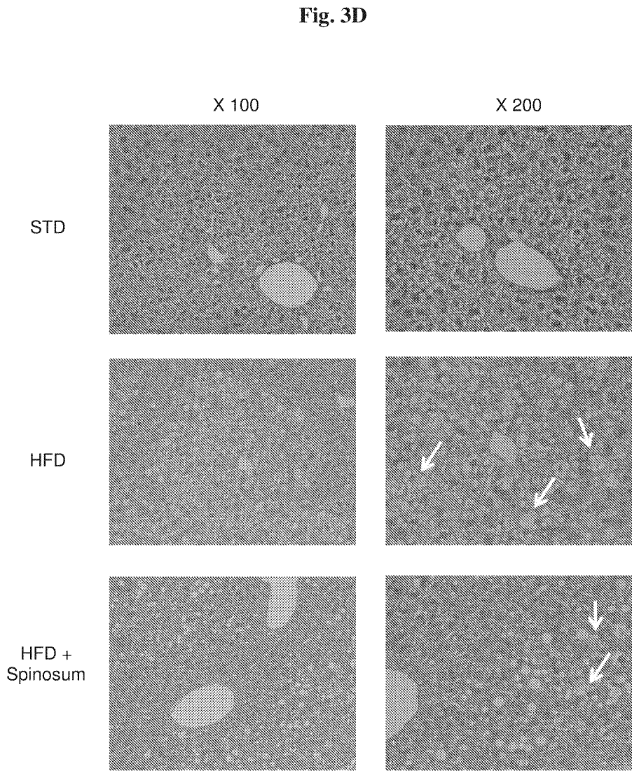

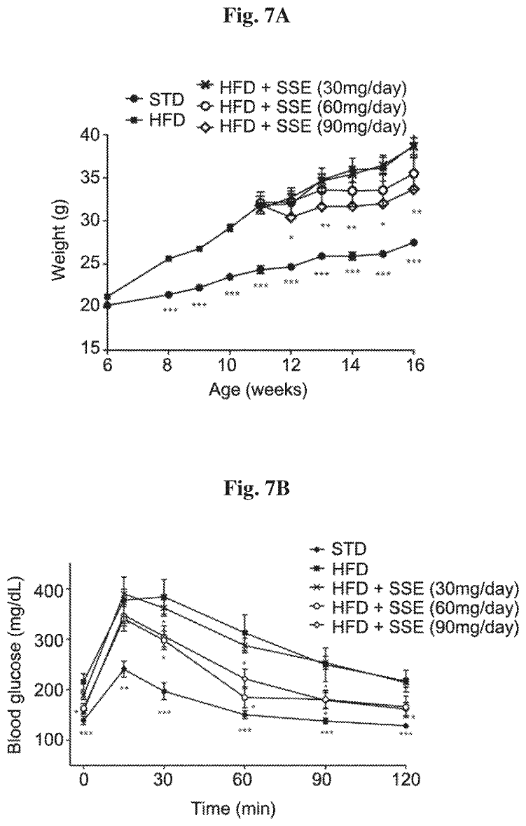

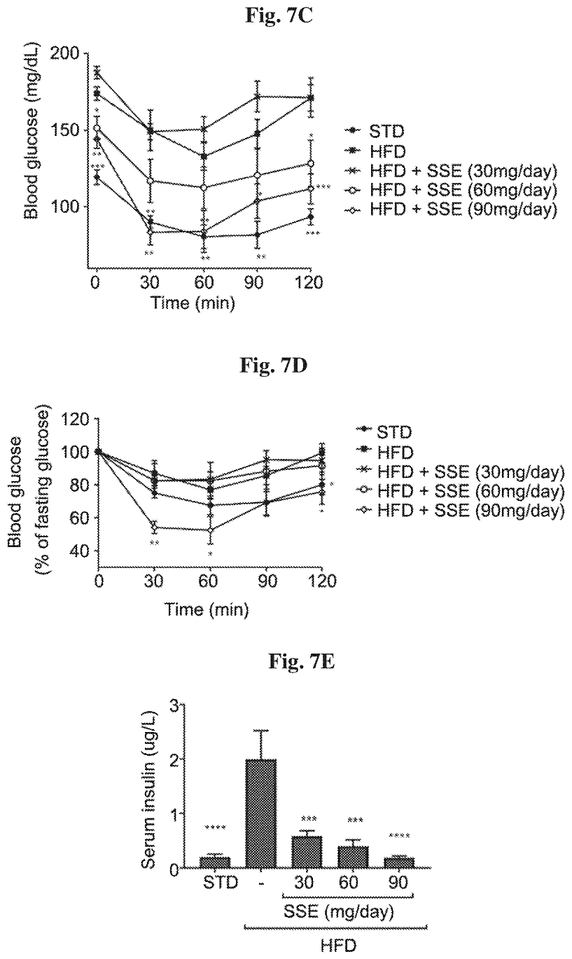

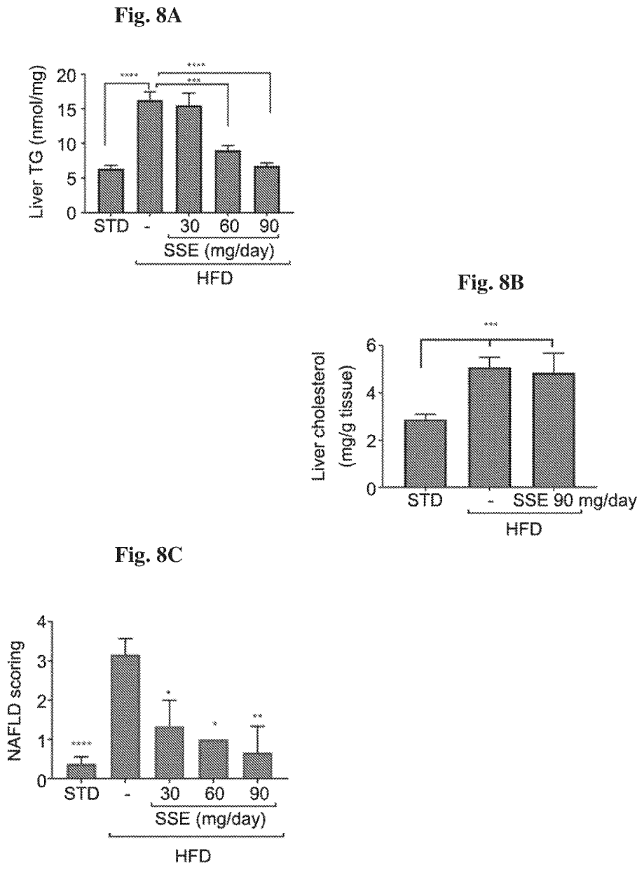

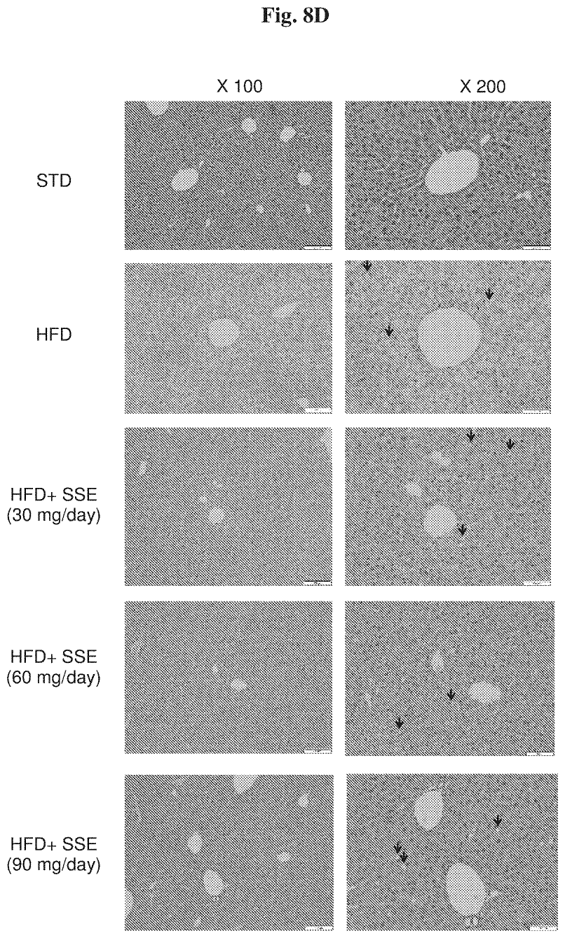

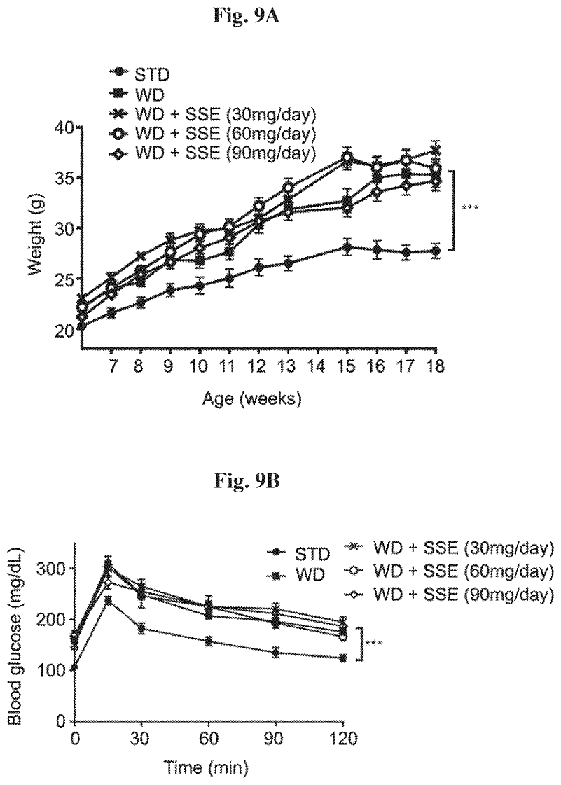

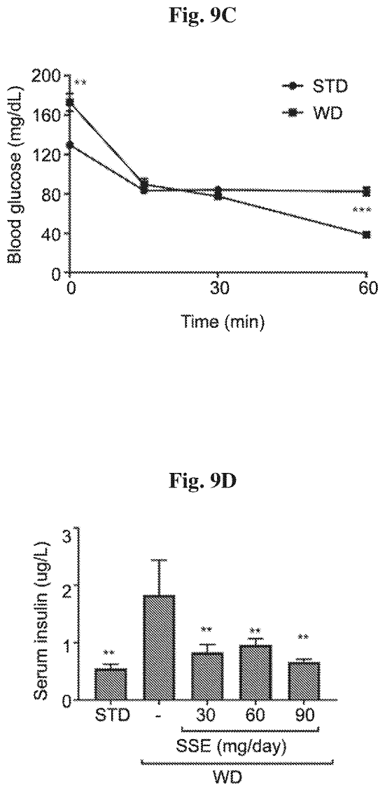

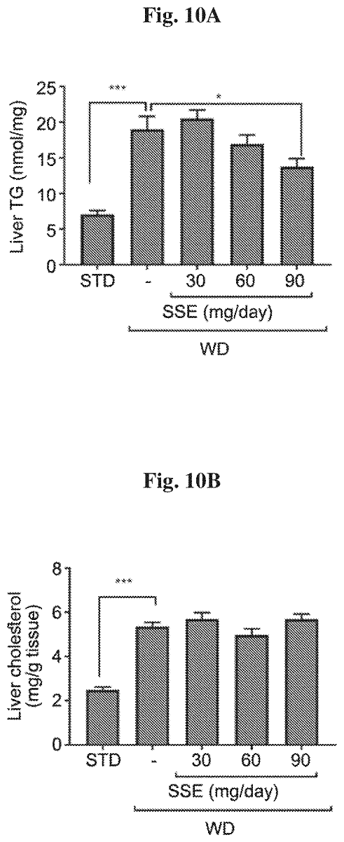

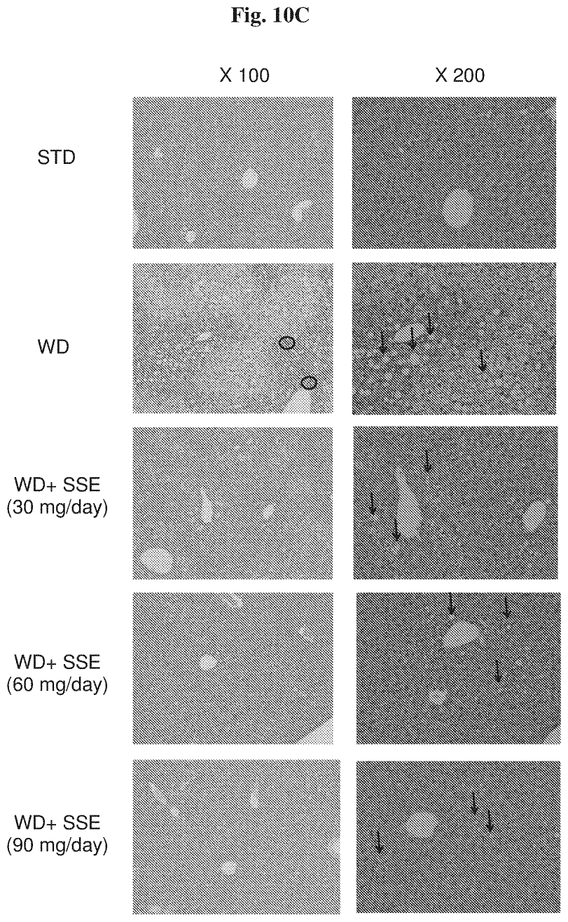

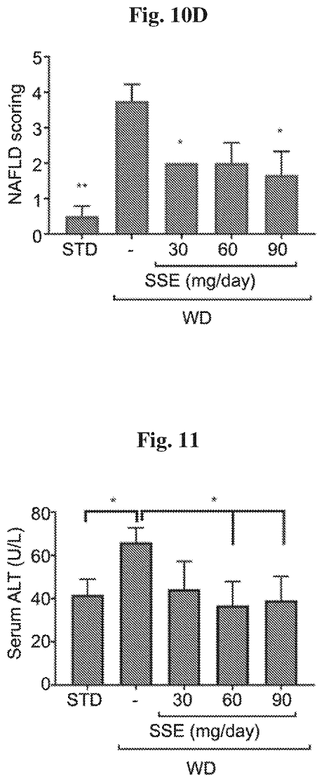

The present invention is based on the finding that an extract of Sarcopoterium spinosum (S. spinosum extract, or SSE) reduced steatosis in mice fed with a high fat diet (HFD, FIGS. 3D and 8D) or a western diet (WD, FIG. 10C). As shown in FIG. 2B, preventive treatment by S. spinosum extract improved signaling in liver, and also resulted in increased expression of lipid metabolism genes that were reduced due to HFD (Example 5). Treatment of HFD or WD-fed mice by the S. spinosum extract of the invention resulted in reduced liver triglycerides (FIGS. 8A for HFD, and 10A for WD), lower scores of NAFLD (FIG. 10D), and reduced hepatic enzymes compared to WD-fed mice (FIG. 11). It is note-worthy that while treatment by SSE improved glucose tolerance in the HFD-fed mice (FIGS. 1 and 7), it seemed not to affect WD-fed mice (FIG. 9B). It is further noted (see Example 11) that the WD-fed mouse model resembles metabolic syndrome in humans, and demonstrates lower levels of fasting glucose and glucose levels following glucose load compared to the HFD-fed mice. Accordingly, the inventors have shown the effect of the S. Spinosum extract on liver function in a model unrelated to diabetes.

Accordingly, in one aspect, the present invention provides a Sarcopoterium spinosum (S. spinosum) extract for use in preventing, treating and/or reducing the risk of developing, fatty liver disease in a subject.

### Embodiments Relating to Diseases, Conditions, and Symptoms:

Fatty liver disease (FLD) is a condition in which fat builds up in the liver. There are two main types of FLD, which are alcoholic FLD (AFLD), caused by excessive alcohol use, and non-alcoholic FLD (NAFLD) which is related to metabolic disorders. The mild form of FLD, fatty liver, manifests histologically by an abnormal retention of lipids within a cell, which is termed steatosis. The mild form of fatty liver may progress to a more severe form, termed steatohepatitis (NASH, for the non-alcoholic case), which is marked by the additional presence of lobar inflammation, hepatocellular ballooning and fibrosis. Liver fibrosis may lead to cirrhosis, which involves a risk for liver failure and hepatocellular carcinoma.

Therefore, in certain embodiments, the S. spinosum extract is used for preventing or treating non-alcoholic fatty liver disease (NAFLD). In certain embodiments, the S. spinosum extract is used for preventing or treating alcoholic fatty liver disease (AFLD).

In certain embodiments, the S. spinosum extract is used for preventing or treating steatohepatitis. In certain embodiments, the S. spinosum extract is used for preventing or treating non-alcoholic steatohepatitis (NASH). In certain embodiments, the S. spinosum extract is used for preventing or treating alcoholic steatohepatitis.

A wide range of diseases and conditions can increase the risk of NAFLD, including: high cholesterol, high levels of blood triglycerides, obesity, hypertension, polycystic ovary syndrome, sleep apnea, type 2 diabetes, hypothyroidism, and hypopituitarism, cardiovascular disease, metabolic syndrome.

Therefore, the S. spinosum extract may be used for preventing or treating fatty liver disease when it appears together with associated conditions selected from, but not being limited to, high cholesterol, high levels of triglycerides in the blood, metabolic syndrome, obesity, hypertension, polycystic ovary syndrome, sleep apnea, hypothyroidism, hypopituitarism, apolipoprotein E-deficiency, and cardiovascular disease.

In addition, the S. spinosum extract may be used for preventing or treating NAFLD both resulting from metabolic disorders such as, e.g., galactosemia, glycogen storage diseases, homocystinuria and tyrosemia, as well as from dietary conditions such as malnutrition, total parenteral nutrition, starvation and over-nutrition, or after exposure to certain drugs, such as amiodarone, antiviral drugs such as nucleoside analogues, aspirin, non-steroidal anti-inflammatory drugs (NSAIDS), estrogens, corticosteroids, methotrexate, tamoxifen, or tetracycline.

The S. spinosum extract may also be used according to the invention for preventing or treating conditions associated with alcohol-related fatty liver or with non-alcoholic fatty liver, such as alcoholic or non-alcoholic hepatitis with liver fibrosis, alcoholic or non-alcoholic steatohepatitis with cirrhosis, or alcoholic or non-alcoholic steatohepatitis with cirrhosis, and hepatocellular carcinoma.

It is appreciated that although fatty liver disease may be associated with diabetes, a subject having NAFLD does not necessarily have diabetes. Accordingly, in some embodiments, the subject does not have diabetes. In some embodiments, the subject is not diabetic or pre-diabetic.

Having diabetes or being diabetic is defined herein as having Hemoglobin A1C>6% or blood glucose level≥125 mg/dL. The term “diabetic” includes individuals having Type I diabetes or Type II diabetes. The term “pre-diabetic” includes individuals having blood glucose level between 100 and 125 mg/dL.

In some embodiments, the treating of fatty liver disease with the S. spinosum extract relates to reducing at least one symptom thereof selected from: intrahepatic triglyceride content, lobar inflammation, hepatocellular ballooning, hepatic fibrosis, hepatic steatosis, and cirrhosis.

### Embodiments Relating to Source, Form and Administration of the Extracts:

Extracts of S. Spinosum may be prepared from the whole plant, as well as from various parts of the plant. Traditionally, the roots of the plant have been used for preparing the extract.

In certain embodiments, the S. spinosum extract is an extract from the roots of the plant. In certain other embodiments, the S. spinosum extract is an extract of the fruits and/or leaves of the plant. In some embodiments, the S. spinosum extract is a whole plant extract.

In some embodiments, the S. spinosum extract is formulated for administration in liquid form, preferably in water. In certain embodiments, the S. spinosum extract is formulated in dry form, for example, as powder, a tablet or a capsule.

In some embodiments, the extract is obtained by boiling of the desired plant part, e.g. root, in water or another suitable solvent, filtering, and optionally lyophilization to get a dry extract. In some embodiments, the extract is obtained by boiling S. Spinosum roots in water, filtering, and optionally lyophilization to get a dry extract.

In certain embodiments, the S. spinosum extract is suitable for oral administration.

The extracts of the present invention may be prepared by preparing tea, infusion, decoction, percolation, or by similar methods of extraction of chemicals from a plant. The extraction may be done in water, or in another appropriate solvent.

In another aspect, the present invention relates to a pharmaceutical composition comprising an extract of Sarcopoterium spinosum as defined above, for use in preventing, treating and/or reducing the risk of developing, fatty liver disease as described above.

The pharmaceutical composition may be formulated with a pharmaceutically acceptable carrier or excipient. In certain other embodiments, the composition is formulated as a herbal composition, such as a herbal composition powder.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

The pharmaceutical preparation for oral administration may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Nutraceuticals are natural, bioactive chemical substances or extracts that provide numerous physiological benefits, including, inter alia, disease prevention and health promotion.

Accordingly, in yet another aspect, the present invention relates to a nutraceutical composition comprising an extract of Sarcopoterium spinosum as defined above, for use in preventing, treating and/or reducing the risk of developing, fatty liver disease as described above.

In certain embodiments, the composition is a nutraceutical composition that may comprise other nutritional or dietary supplements such as vitamins, and/or one or more excipients that may be pharmaceutically acceptable or nutraceutical carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient. In certain embodiments, the composition is formulated as an oral formulation that may be liquid or solid, e.g., in the form of tablets, lozenges, capsules, syrup and the like.

In some embodiments, the pharmaceutical or the nutraceutical composition of the invention is used for preventing or treating non-alcoholic fatty liver disease (NAFLD). In certain embodiments, the pharmaceutical or the nutraceutical composition of the invention is used for preventing or treating alcoholic fatty liver disease (AFLD). In certain embodiments, the pharmaceutical or the nutraceutical composition of the invention is used for preventing or treating steatohepatitis. In certain embodiments, the pharmaceutical or the nutraceutical composition of the invention is used for preventing or treating non-alcoholic steatohepatitis (NASH). In certain embodiments, the pharmaceutical or the nutraceutical composition of the invention is used for preventing or treating alcoholic steatohepatitis. In some embodiments, the extract is an extract from the root of S. spinosum. In some embodiments, the extract is in a liquid form. In some embodiments, the extract is in a dry form, such as powder, a tablet or a capsule.

According to another aspect, the present invention provides a method for preventing, treating and/or reducing the risk of developing fatty liver disease in a subject, comprising administering to said subject an extract of Sarcopoterium spinosum according to the invention as described above.

The determination of the doses of the active ingredient to be used for human use is based on commonly used practices in the art, and will be finally determined by physicians in clinical trials. An expected approximate equivalent dose for administration to a human can be calculated based on the in vivo experimental evidence disclosed herein below, using known formulas (e.g. Reagan-Show et al. (2007) Dose translation from animal to human studies revisited. The FASEB Journal 22:659-661). According to this paradigm, the adult human equivalent dose (mg/kg body weight) equals a dose given to a mouse (mg/kg body weight) multiplied with 0.081.

With regard to the daily dose of the dry S. spinosum extract to be administered to humans, based on the experiments in mice disclosed in the present application (30, 60, or 90 mg/day per mouse), the daily dose may vary in certain embodiments between doses of about 2.5 g to about 25 g, and in other embodiments between doses of between about 4 g to about 15 g. Alternatively, the daily dose of the dry S. spinosum extract for a human subject per kg is between about 40 mg/kg to about 400 mg/kg or between about 70 mg/kg to about 250 mg/kg. The daily dose of the S. spinosum extract may be administered once or more daily, as needed.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The term “treating” or “treatment” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease. This term also includes reversing or slowing the progression of disease activity or the medical consequences of the disease.

The term “preventing” as used herein relates to suspending, postponing, delaying or completely abolishing the appearance of a certain disorder, disease, or condition, or the appearance of symptoms associated with a certain disorder, disease, or condition. Further, the terms “preventing” and “prevention” refer to prophylactic use to reduce the likelihood of a disease, disorder, or condition to which such terms apply, or one or more symptoms of such disease, disorder, or condition. It is not necessary to achieve a 100% likelihood of prevention; it is sufficient to achieve at least a partial effect of reducing the risk of acquiring such disease, disorder, or condition.

The term “about” as used herein means that values that are 10% more or less than the indicate value are also intended to be included.

It is appreciated that all the embodiments of the first aspect, which relates to the extract, even if not explicitly indicated, also apply to the aspects relating to the pharmaceutical or nutraceutical composition, and the method. These embodiments include at least the embodiments relating to diseases, conditions, and symptoms, and embodiments relating to the source, form, and administration of extract.

The invention will now be demonstrated by the following non-limiting examples.

### EXAMPLES

### Methods

S. spinosum Extract Preparation

100g fresh S. spinosum roots were cut into small pieces and boiled in 1 L of water for 30 min. The solutions were left for 3 h and the red supernatants were filtered to a sterile bottle without disturbing the pellet, and kept at 4° C. In the experiments indicated, extract was dried by lyophilization, giving a yield of 7 gr/L.

Study Design

Prevention Protocol of Hepatic Steatosis:

The study was carried out in accordance with the recommendations in the Guide for the Care and Use of Laboratory Animals of the National Institutes of Health. The Animal House in Ariel University operates in compliance with the rules and guidelines of the Israel Council for Research in Animals, based on the US NIH Guide for the Care and Use of Laboratory Animals. The mice were housed in an animal laboratory with a controlled environment of 20-24° C., 45-65% humidity, and a 12 h light/dark cycle Animals had been anesthetized by ketamine+xylazine as required, and all efforts were made to minimize suffering.

The study was performed on a model of diet-induced glucose intolerance, using high fat diet-fed C57bl/6 mice (HFD, 60% of total calories derived from fatty acids, 18.4% from proteins, and 21.3% from carbohydrates, Envigo, Teklad TD.06414). C57B1/6J mice were purchased from Envigo (Israel). 6 week old male C57bl/6 mice were separated into 3 treatment groups, 8-10 mice each, as follows: control mice fed with standard (STD) diet (18% of total calories derived from fat, 24% from proteins, and 58% from carbohydrates. Harlan, Teklad TD.2018) or HFD, and HFD-fed mice supplemented with S. spinosum extract (SSE). SSE (30 mg/day dry material) was administered daily in the drinking water starting at age 6 weeks. Body weight was measured once a week. At age of 17 weeks, mice were anesthetized using ketamine+xylazine and euthanized by terminal bleeding followed by cervical dislocation.

Blood was collected from the heart and serum was prepared. Serum insulin was measured by immunoassay, using a commercial ELISA kit (Mercodia, Sweden). Livers were perfused and both liver and soleus muscle were isolated. In order to follow insulin-induced protein phosphorylation in liver and skeletal muscle, in some of the mice (n=5), insulin was injected (0.75 mU/g body weight) 15 min before killing the animal. Liver and muscle were snap frozen in liquid nitrogen, and preserved in −80° C. for later protein and RNA extraction. Liver parts were saved in 4% paraformaldehyde for histological analyses.

Treatment Protocol of Hepatic Steatosis:

The study was performed using the same model of diet-induced glucose intolerance as described above, using high fat diet-fed C57bl/6 mice. For these experiments, 6 week old male mice were given either STD or HFD. At age of 10 weeks, HFD-fed mice were separated into treatment groups, 8-10 mice each, as follows: HFD, and HFD-fed mice supplemented with S. spinosum extract (SSE) at 3 different doses (30, 60 and 90 mg/day). While the diet regime was given as early as the age of 6 week, SSE was supplemented to the diet at age of 10 weeks. Body weight was measured once a week. At age of 17 weeks, mice were anesthetized using ketamine+xylazine and euthanized by terminal bleeding followed by cervical dislocation. Blood was collected from the heart and serum was prepared. Serum insulin was measured by immunoassay, using a commercial ELISA kit (Mercodia, Sweden). Liver was snap frozen in liquid nitrogen, and preserved in −80° C. for later lipid extraction. Liver parts were saved in 4% paraformaldehyde for histological analyses.

NASH Model

Fructose consumption is necessary for the progression of NAFLD from the 1^{st} stage of steatosis to the 2^{nd} stage of steatohepatitis (NASH). NASH is characterized by hepatocellular steatosis with additional pathological features, such as hepatic inflammation and sinusoidal collagen formation representing initiation of liver fibrosis. There is a reduction in hepatic function at this stage, as can be evaluated by measuring liver enzymes in the plasma. Western diet-fed C57bl/6 mice with access to fructose in drinking water (ad libitum) was validated as a model for NASH.

The model is developed by feeding the mice with western diet (WD) which contains 42% fat, 42.7% carbohydrate, and 15.2% protein. Fructose was added to the drinking water at a concentration of 42 g/L. Mice were randomly divided into STD-fed (1 group) or WD-fed (4 groups) for 4 weeks (from the age of six weeks until the age of 10 weeks). After 4 weeks of STD or WD diet, groups 3, 4 and 5 were given 30, 60 or 90 mg/day, respectively, of a dried extract of S. spinosum for additional 8 weeks. Body weight was measured once a week. Mice were sacrificed at age of 20 weeks: mice were anesthetized by ketamin/xylazine and terminal bleeding is performed. Livers were perfused and saved for later histological and biochemical analyses. Serum was prepared and saved at −80° C. for later analyses of lipids, cytokines and hepatic enzymes.

Glucose Tolerance Test (GTT)

Intraperitoneal glucose tolerance test (GTT) was performed in C57bl/6 mice at age of 15 weeks. Mice were injected with 1.5 mg glucose/g body weight after 6 h fast. Blood glucose was determined from tail blood using the ACCU-CHEK Go glucometer (Roche, Germany).

Insulin Tolerance Test (ITT)

Insulin tolerance test (ITT) was performed at age 15 weeks following a 6 h fast. Glucose was measured following intraperitoneal insulin injection (0.5 U/kg).

Analysis of mRNA Expression by Real-Time PCR

Total RNA was extracted from liver using RNeasy lipid tissue mini kit (Qiagen), and TRI reagent (Molecular Research Center, Inc. Cincinnati, Ohio), respectively, according to manufacturers' instruction. 3-4 μg of total RNA were reverse transcribed by oligo dT priming (Stratascript 5.0 multi-temperature reverse transcriptase, Stratagene) according to the manufacturers' instructions. Real-time PCR amplification reactions were performed using SYBR Fast Universal Ready-mix Kit (Kappa biosystems), by the MxPro QPCR instrument (Stratagene). Primers for real time PCR reactions were synthesized by Sigma, Israel. Primer sequences were as shown in Table 1:

TABLE 1
primers sequences

| Gene | Forward | ID | Reverse | ID |
|---|---|---|---|---|
| PPARα | ATGCCAGTACTGCC | 1 | CCGAATCTTTCAGGTCGTGT | 2 |
| | GTTTTC | | | |
| PPARγ | CAGGCCTCATGAAG | 3 | ACCCTTGCATCCTTCACAAG | 4 |
| | AACCTT | | | |
| SREBP2 | AGAGGCGGACAACA | 5 | ACGCCAGACTTGTGCATCTT | 6 |
| | CACAAT | | | |
| SREBP1c | AAGAGCCCTGCACT | 7 | CCACAAAGAAACGGTGACCT | 8 |
| | TCTTGA | | | |
| HSL | TGCTCTTCTTCGAG | 9 | TCTCGTTGCGTTTGTAGTGC | 10 |
| | GGTGAT | | | |
| ACC1 | CATGAACACCCAGA | 11 | ATTTGTCGTAGTGGCCGTTC | 12 |
| | GCATTG | | | |
| FAS | TTGCTGGCACTACA | 13 | AACAGCCTCAGAGCGACAAT | 14 |
| | GAATGC | | | |
| AdipoR1 | TCGTGTATAAGGTC | 15 | GCAGATGTGTCCAGATGTTG | 16 |
| | TGGGAG | | | |
| AdipoR2 | CCCAGGAAGATGAA | 17 | TTAAGCCAATCCGGTAGCAC | 18 |
| | GGGTTT | | | |
| HPRT | TGTTGTTGGATATG | 19 | TTGCGCTCATCTTAGGCTTT | 20 |
| | CCCTTG | | | |

**ID: SEQ ID NO.

Hepatic Triglyceride (TG) Levels

100 mg of liver was homogenized in 1 ml containing 5% NP-40 in water. The samples were twice heated to 80-100° C. for 5 min and cooled to room temperature. The sample was centrifuged for 2 min and the supernatant was used for triglycerides (TG) analysis. TGs were measured using Triglyceride Quantification Kit (ab65336, Abcam, Cambridge, UK) according to manufacturer's instruction.

Hepatic Cholesterol Levels

10 mg of liver were homogenized in a solution of Chloroform:Isopropanol:NP-40 (7:11:0.1). The organic phase was collected and vacuum dried for about 2 h, and resuspended in Cholesterol Assay Buffer supplied by Abcam. Total cholesterol was measured by Cholesterol/Cholesteryl Ester quantitation kit (ab65359, Abcam, Cambridge, UK) according to manufacturer's instruction.

Hepatic Glycogen Content

10 mg of liver were homogenized in glycogen hydrolysis buffer supplied by Abcam. The sample was centrifuged for 5 min and the supernatant was used for glycogen analysis using Glycogen Assay Kit II (ab65620, Abcam, Cambridge, UK) according to manufacturer's instruction.

Histochemistry

Livers were perfused, isolated, fixed in 4% paraformaldehyde and embedded in paraffin. Consecutive 4 μm sections were cut and stained with hematoxylin and eosin (H&E). The presence of inflammation and steatosis score was blinded evaluated by a pathologist. Scoring of liver sections was adapted from Liang W. et al., Establishment of a General NAFLD Scoring System for Rodent Models and Comparison to Human Liver Pathology, PLoS ONE 9(12): e115922. Evaluation was done with Olympus light microscope BX43, Olympus digital camera DP21 with Olympus cellSens Entry 1.13 software.

Serum ALT (Alanine Transaminase) and AST (Aspartate Transaminase)

Serum ALT and AST levels were measured in fresh samples using the Alanine Transaminase and Aspartate Aminotransferase activity assay kits (ab105134 and ab105130, respectively, Abcam, Cambridge, UK), respectively, according to manufacturer's instruction.

Statistical Analysis

Values are presented as mean±SEM. Statistical differences between the treatments and controls were tested by unpaired two-tailed Student's t-test or one-way analysis of variance (ANOVA), followed by Bonferroni's post hoc testing, when appropriate. Analysis was performed using the GraphPad Prism 5.0 software. A difference of p<0.05 or less in the mean values was considered statistically significant.

### Example 1

### The Effect of S. spinosum Extract in Solution on a Mouse Model of Steatosis (Prevention Protocol)

The study was performed on a model of diet-induced glucose intolerance, using high fat diet (HFD)-fed C57bl/6 mice (HFD, 60% of total calories derived from fatty acids, 18.4% from proteins and 21.3% from carbohydrates, Envigo Teklad diet TD.06414). Six weeks old male mice were separated into 3 treatment groups, 8-10 mice each, as follows: 1) control C57bl/6 mice fed with standard diet (STD, 18% of total calories derived from fat, 24% from proteins and 58% from carbohydrates. Envigo Teklad diet TD.2018); 2) HFD-fed mice; and 3) HFD-fed mice where the diet was supplemented with S. spinosum extract which was administered daily in the drinking water starting at age of 6 weeks.

Body weight was measured once a week. At age 16 weeks, mice were anesthetized using ketamine+xylazine and euthanized by terminal bleeding followed by cervical dislocation. Blood was collected from the heart and serum was prepared. Livers were perfused and isolated. Liver was snap-frozen in liquid nitrogen, and preserved in −80° C. for later lipids, protein and RNA extraction. Parts of the livers were saved in 4% paraformaldehyde for histological analyses.

### Example 2

### S. spinosum Improves Glucose Tolerance in High Fat Diet (HFD)-Fed Mice (Prevention Protocol)

The effects of S. spinosum root extract on body weight and glucose homeostasis was followed. A significant increase in body weight of the HFD-fed groups (FIG. 1A) was demonstrated. Food consumption and drinking habits were measured, demonstrating lower food consumption in HFD-fed groups and no difference between all 3 groups in drinking habits (data not shown). Body weight was higher in HFD-fed mice, with no effect of S. spinosum on the rate of body weight accumulation. Fasting blood glucose and glucose disposal following intraperitoneal glucose load was altered in HFD-fed mice. S. spinosum extract did not affect fasting glucose levels but improved glucose disposal of the HFD-fed mice (FIG. 1B), an effect that is accompanied by reduced fasting serum insulin levels (FIG. 1C). These results suggest a positive effect of S. spinosum extract on HFD-fed mice.

### Example 3

### S. spinosum Improves Insulin Signaling in Liver of HFD-Fed Mice (Prevention Protocol)

In order to determine whether the improvement in glucose tolerance in glucose intolerant HFD-fed mice is mediated by elevated activation of the insulin signaling cascade, the phosphorylation of key proteins mediating insulin signal transduction was followed in liver (FIG. 2A). A significant increase in insulin-induced phosphorylation of insulin receptor (IR) and GSK-3β in liver of S. spinosum treated, HFD-fed mice was found (FIG. 2B). These findings support the suggestion that S. spinosum extract improved metabolic hepatic function.

### Example 4

### Effect of S. spinosum Extract on Hepatic Glycogen and Lipid Storage (Prevention Protocol)

Glycogen levels were elevated in HFD-fed mice supplemented with S. spinosum extract (FIG. 3A). This increase in carbohydrate stores is in accord with the high phosphorylation level of GSK3β demonstrated in these mice.

On the other hand, triglyceride and cholesterol levels were not affected by S. spinosum treatment (FIGS. 3B, C); both non-treated and S. spinosum-treated groups of HFD-fed mice had elevated lipid levels in the liver. On the other hand, histological evaluation of the severity of steatosis in the livers of HFD-fed mice with or without S. spinosum supplementation clearly indicates lower steatosis state in S. spinosum treated mice, similar to the control (FIG. 3D).

### Example 5

### Effect of S. spinosum Extract on Glucose and Lipid Metabolism Genes in Liver of HFD-Hed Mice

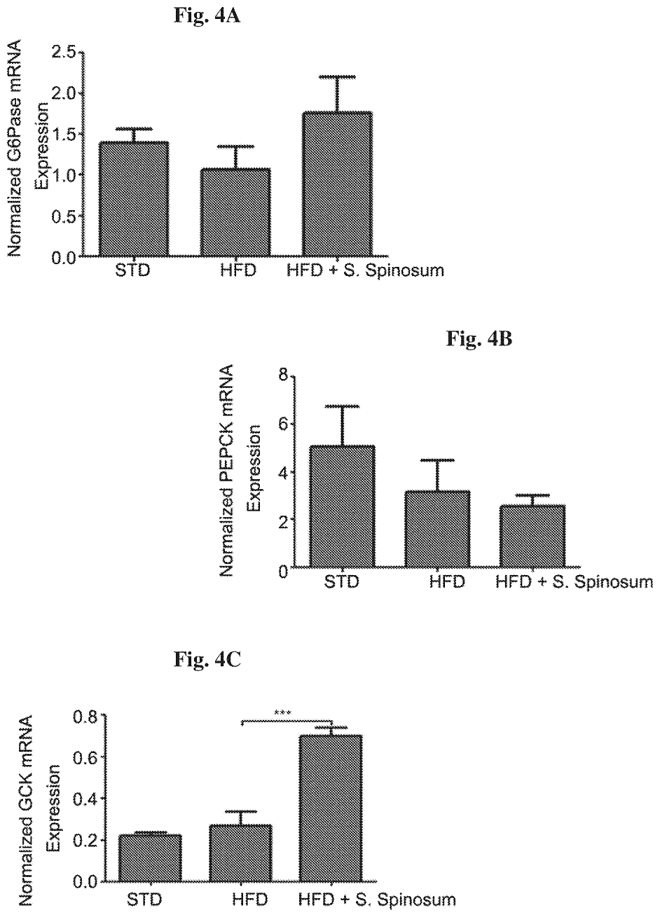

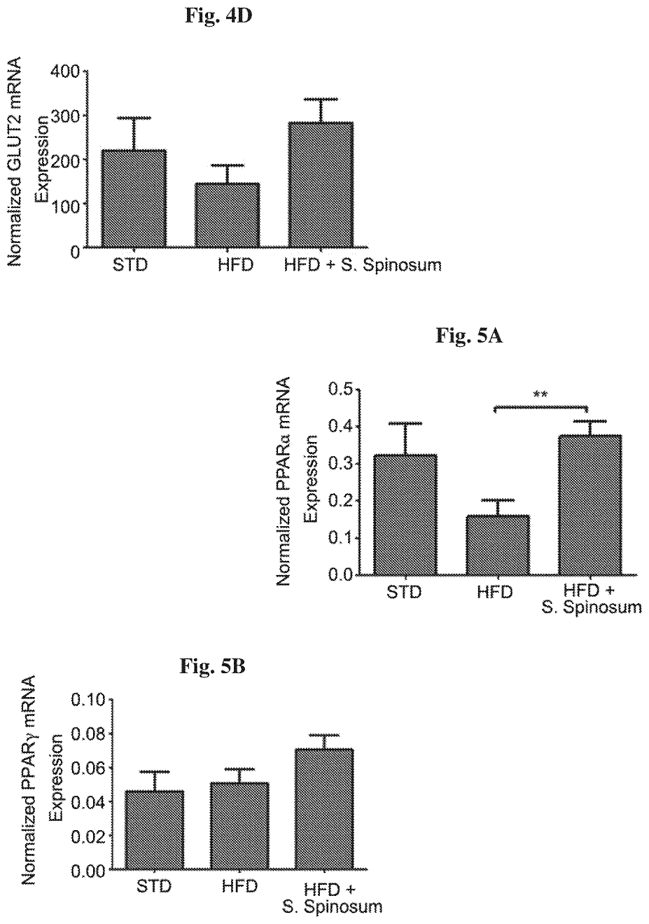

The results so far suggest an improved function of the liver following S. spinosum supplementation. Since the HFD-fed mice demonstrate the lower stage of non-alcoholic fatty liver disease (NAFLD), which includes hepatic steatosis with the absence of inflammation, fibrosis and hepatic dysfunction, in order to support the results suggesting beneficial effects of the extract on liver, we measured the liver mRNA expression of several genes involved in glucose and lipid metabolism. While mRNA expression of the gluconeogenic genes G6Pase and PEPCK was not affected by the diet or by S. spinosum treatment (FIG. 4A, B, respectively), GCK expression was increased (FIG. 4C). The GCK gene encodes for glucokinase, an enzyme that phosphorylates glucose, thereby maintaining the concentration gradient for glucose and facilitating its transport inside the hepatocyte. Glut2 mRNA expression was not significantly affected by the diet or the extract (FIG. 4D).

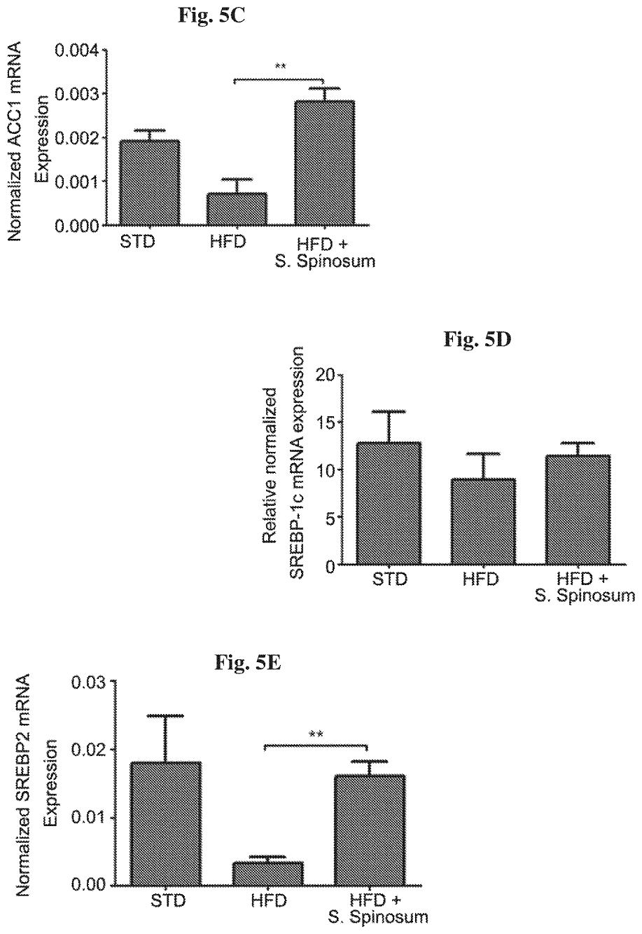

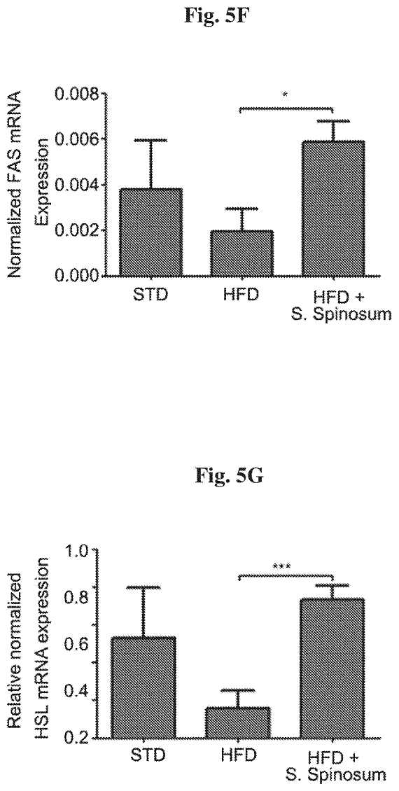

Regarding the expression of genes regulating lipid metabolism, while some genes were not affected by the diet and the extract (FIGS. 5B, D), several other genes were downregulated by HFD, including lipogenic genes (ACC1, SREBP2 and FAS, FIGS. 5C, E, and F, respectively) as well as genes involved in lipolysis (HSL, FIG. 5G). The expression of PPARα, a key master in lipid metabolism and oxidation was also reduced in HFD-fed mice (FIG. 5A). The expression of all of the genes the expression of which had been reduced by the HFD diet was increased by treating with S. spinosum, suggesting that this extract normalizes the profile of gene expression and metabolic function of the liver despite lipid oversupply by diet.

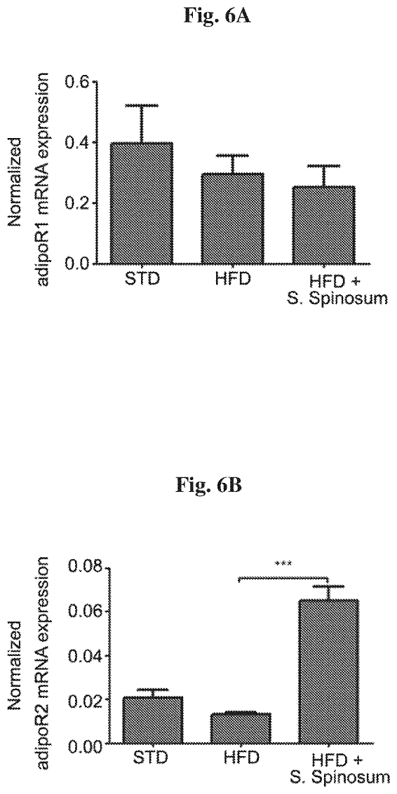

In accord with these results, mRNA expression of AdipoR2, the adiponectin receptor predominantly expressed in the liver, was increased in HFD-fed mice treated by S. spinosum (FIG. 6B). Adiponectin is known to decrease hepatic insulin resistance and to attenuate liver inflammation and fibrosis, thus the increase in the expression of its receptor suggest an increased activation of adiponectin function in the liver of S. spinosum-supplemented mice.

### Example 6

### Effect of a Dry S. spinosum Extract on a Mouse Model of Steatosis (Treatment Protocol)

6 weeks old male mice as described in Example 1 were randomly divided into STD-fed (group 1) and HFD-fed (groups 2-5), and are fed based on the chosen diet for 4 weeks (from the age of six weeks until the age of 10 weeks). After 4 weeks of STD or HFD, groups 3, 4 and 5 are given 30, 60 or 90 mg/day, respectively, of a dried extract of S. spinosum for additional 7 weeks.

Body weight was measured once a week. At age 17 weeks, mice were anesthetized using ketamine+xylazine and euthanized by terminal bleeding followed by cervical dislocation. Blood was collected from the heart and serum was prepared. Livers were perfused and isolated. Liver was snap-frozen in liquid nitrogen, and preserved in −80° C. for later lipids, protein and RNA extraction. Parts of the livers were saved in 4% paraformaldehyde for histological analyses.

### Example 7

### S. spinosum Extract Improves Glucose Tolerance in High Fat Diet (HFD)-Fed Mice (Treatment Protocol)

The effects of S. spinosum root extract on body weight and glucose homeostasis was followed. A significant increase in body weight of the HFD-fed groups (FIG. 7A) was demonstrated. Food consumption and drinking habits were measured, demonstrating lower food consumption in HFD-fed groups and no difference between all 5 groups in drinking habits (data not shown). HFD induced a significant increase in body weight, compared to STD. Treatment by SSE consumed at the highest dose (90 mg/day) lead to lower body weight accumulation compared to their HFD-fed littermates (FIG. 7A). Fasting blood glucose and glucose disposal following intraperitoneal glucose load was altered in HFD-fed mice. S. spinosum extract (60, 90 mg/day) improved both fasting and glucose disposal of the HFD-fed mice (FIG. 7B). Insulin resistance was developed in HFD-fed mice, as demonstrated by an impaired insulin tolerance test (FIGS. 7C and 7D) and elevated serum insulin (FIG. 7E), while SSE supplementation improved the sensitivity to the hormone, leading to improved response to insulin injection (FIGS. 7C and 7D) and reduced serum level of insulin (FIG. 7E). These results suggest a positive effect of S. spinosum extract on glucose tolerance and insulin sensitivity in HFD-fed mice

### Example 8

### Effect of S. spinosum Extract on Hepatic Lipid Storage (Treatment Protocol)

Triglyceride levels were completely normalized by S. spinosum treatment in a dose-dependent manner (FIG. 8A). Hepatic total cholesterol was higher in HFD-fed mice and was not affected by SSE supplementation (FIG. 8B). H&E staining of liver was performed. A histological evaluation of the severity of steatosis in the livers of HFD-fed mice with or without S. spinosum supplementation done by independent pathologist (Patho-Lab Diagnostics Ltd, Israel) demonstrated a reduction in NAFLD scoring in SSE-treated mice, in all doses given (FIG. 8C). HFD feeding induced severe hepatic steatosis, covering over 66% of hepatic area. Lipid droplets can be seen in almost all hepatocytes (FIG. 8D, arrows point to representative steatotic hepatocytes). An improvement in liver steatosis is demonstrated in mice treated with 30 and 60 mg/day S. spinosum extract, showing much smaller lipid droplets, while an almost complete normalization of liver morphology was found in HFD-fed mice treated by 90 mg/day SSE, where only a few and sporadic lipid droplets were detected, while hepatocyte morphology was normal.

### Example 9

### Effect of S. spinosum Extract on a Model of Steatohepatitis

Fructose consumption is necessary for the progression of NAFLD from the 1^{st} stage of steatosis to the 2^{nd} stage of steatohepatitis (NASH). NASH is characterized by hepatocellular steatosis with additional pathological features, such as hepatic inflammation and sinusoidal collagen formation representing initiation of liver fibrosis. There is a reduction in hepatic function in this stage, as can be evaluated by measuring liver enzymes in the plasma. Western diet-fed c57bl/6 mice with the access to fructose in drinking water (ad libitum) develop all pathological characteristics.

The model was developed by feeding the mice with western diet (WD) which contains 42% fat, 42.7% carbohydrate, and 15.2% protein. Fructose was added to the drinking water at a concentration of 42 g/L. Mice are randomly divided into STD-fed (1 group) or WD-fed (4 groups) for 4 weeks (from the age of six weeks until the age of 10 weeks). After 4 weeks of WD-feeding, groups 3, 4 and 5 were given 30, 60 or 90 mg/day, respectively, of a dried extract of S. spinosum for additional 8 weeks, with continuous WD feeding. Body weight was measured once a week. Mice were sacrificed at age 20 weeks: mice were anesthetized by ketamin/xylazine and terminal bleeding was performed. Livers were perfused and saved for later histological, biochemical and molecular (mRNA expression) analyses. Serum was prepared and saved at −80° C. for later analyses of lipids and hepatic enzymes.

### Example 10

### S. spinosum Does Not Improve Glucose Tolerance in a Model of Steatohepatitis

The effects of treatment with S. spinosum root extract on body weight and glucose homeostasis was followed. A significant increase in body weight of the WD-fed groups (FIG. 9A) was demonstrated. Food consumption and drinking habits were measured, demonstrating lower food consumption in WD-fed groups and no difference between all 5 groups in drinking habits (data not shown). Body weight was higher in WD-fed mice, with no effect of S. spinosum on the rate of body weight accumulation. Fasting blood glucose and glucose disposal following intraperitoneal glucose load were much lower in the WD-fed mouse model of NASH than in the HFD model (average fasting glucose of 159 mg/dL vs. 217 mg/dL in WD and HFD-fed mice respectively, and max glucose level measured 30 min following glucose load of 309 mg/dL vs 416 mg/dL in WD and HFD-fed mice respectively). It can be seen that S. spinosum extract did not affect fasting glucose or glucose disposal of the WD-fed mice (FIG. 9B).

In addition, insulin load, performed as part of the insulin tolerance test, at age of 10 weeks old, induced a severe hypoglycemia (FIG. 9C), indicating that insulin resistance is not developed in this model of NASH. The hypoglycemic response to insulin might be attributed to lower rate of hepatic insulin clearance as a result of impaired hepatic function. This hypothesis of impaired insulin clearance is supported by an increased serum insulin in WD-fed mice (FIG. 9D), which is suggested to reflect defects in insulin clearance rather than high insulin secretion (Bril et al., 2014; Livero et al., 2016). This elevated serum insulin was corrected by SSE treatment. Because of the hypoglycemic response, the insulin tolerance test was not performed in this model again, thus no data are available on mice at age of 18 weeks old following SSE treatment. These results suggest that the glucose intolerance and insulin resistance are not a dominant pathology in this model.

### Example 11

### Effect of S. spinosum Extract on Hepatic Lipid Storage (NASH Protocol)

Hepatic triglyceride (TG) and cholesterol levels were increased in WD-fed mice. SSE (90 mg/day) reduced the severity of TG accumulation in the liver, while cholesterol levels were not affected (FIGS. 10A, B); Histological evaluation of the severity of steatosis in the livers of WD-fed mice with or without S. spinosum treatment clearly indicates lower NAFLD score in S. spinosum treated mice (FIG. 10C and D). As seen from FIG. 10C, while WD-fed mice developed a severe hepatic steatosis, covering over 66% of liver samples, SSE reduced the severity of steatosis to a level recognized as mild, covering 5-33% of liver sample, in all doses used in this study (arrows point to representative steatotic hepatocytes). In addition, foci of inflammation were detected in liver of WD-fed mice (marked in circles), but not in S. spinosum treated mice.

### Example 12

### Effect of S. spinosum Extract on Serum Level of Hepatic Enzymes (NASH Protocol)

Serum ALT (alanine transaminase) and AST (aspartate transaminase) levels were measured. AST levels were not affected by the WD regime in all experimental groups (data not shown). An increase in serum ALT was induced by WD, which was completely corrected by SSE treatment, suggesting that SSE eliminate the hepatic damage induced by the WD (FIG. 11).

### Example 13

### Effect of S. spinosum Extract on Liver Function in a Mouse Model of AFLD

8 weeks old C57BL/6 female mice are subjected to 6 weeks of chronic ethanol feeding (5%, v/v), with or without the addition of S. spinosum dried extract at 30, 60 and 90 mg/day. Control mice are fed dextran-maltose instead of the ethanol for replacing the ethanol calories. Body weight and serum levels of ALT, AST, TG, and cholesterol are measured in the end of the 6 weeks ethanol feeding, as well as a histological evaluation and scoring of liver steatosis, as detailed above.

It is expected that mice fed with ethanol will exhibit steatosis, and elevated liver enzymes compared to mice not fed with ethanol. It is further expected that mice treated with SSE will exhibit less steatosis and lower liver enzyme levels compared to untreated mice.

### REFERENCES

Bedogni G, Miglioli L, Masutti F, Tiribelli C, Marchesini G, Bellentani S. Prevalence of and risk factors for nonalcoholic fatty liver disease: the Dionysos nutrition and liver study. Hepatology 2005 ;42(1):44-52.

Bertola A, Mathews S, Ki S H, Wang H, Gao B. Mouse model of chronic and binge ethanol feeding (the NIAAA model). Nature protocols 2013;8(3):627-37.

Bril F, Lomonaco R, Orsak B, Ortiz-Lopez C, Webb A, Tio F, Hecht J, Cusi K. Relationship between disease severity, hyperinsulinemia, and impaired insulin clearance in patients with nonalcoholic steatohepatitis. Hepatology 2014;59(6):2178-87.

Livero F A, Acco A. Molecular basis of alcoholic fatty liver disease: From incidence to treatment. Hepatology research: the official journal of the Japan Society of Hepatology 2016;46(1):111-23.

1-21. (canceled)

22. A method for treating, preventing, and/or reducing the risk of developing, fatty liver disease in a subject, comprising administering to said subject a therapeutically effective amount of a Sarcopoterium spinosum extract (SSE).

23. The method according to claim 22, wherein the fatty liver disease is non-alcoholic fatty liver disease (NAFLD).

24. The method according to claim 22, wherein the fatty liver disease is non-alcoholic steatohepatitis (NASH).

25. The method according to claim 22, wherein the subject does not have diabetes.

26. The method according to claim 22, wherein said treating comprises reducing at least one symptom of fatty liver disease, selected from intrahepatic triglyceride content, lobar inflammation, hepatocellular ballooning, hepatic fibrosis, hepatic steatosis, and cirrhosis.

27. The method according to claim 22, wherein the SSE is an extract from the root of S. spinosum.

28. The method according to claim 22, wherein the SSE is in liquid form.

29. The method according to claim 22, wherein the SSE is in a dry form, such as powder, a tablet or a capsule.

30. The method according to claim 22, comprising the step of obtaining the SSE by boiling a desired Sarcopoterium spinosum plant part in water, filtering, and optionally lyophilizing

31. The method of claim 22, wherein the SSE is comprised in a pharmaceutical composition.

32. The method of claim 22, wherein the SSE is comprised in a nutraceutical composition.

33. The method of claim 32, wherein the nutraceutical composition further comprises other nutritional or dietary supplements and/or one or more excipients that may be pharmaceutically acceptable or nutraceutical carriers, diluents, adjuvants, excipients, or vehicles.

34. The method of claim 33, wherein the nutraceutical composition is formulated for oral administration in the form of a tablet, a capsule, a pill, lozenge or syrup.


SEQUENCE LISTING (20 sequences)

SEQ ID NO: 1 - DNA, 20 bp, artificial sequence
  synthetic
atgccagtactgccgttttc

SEQ ID NO: 2 - DNA, 20 bp, artificial sequence
  synthetic
ccgaatctttcaggtcgtgt

SEQ ID NO: 3 - DNA, 20 bp, artificial sequence
  synthetic
caggcctcatgaagaacctt

SEQ ID NO: 4 - DNA, 20 bp, artificial sequence
  synthetic
acccttgcatccttcacaag

SEQ ID NO: 5 - DNA, 20 bp, artificial sequence
  synthetic
agaggcggacaacacacaat

SEQ ID NO: 6 - DNA, 20 bp, artificial sequence
  synthetic
acgccagacttgtgcatctt

SEQ ID NO: 7 - DNA, 20 bp, artificial sequence
  synthetic
aagagccctgcacttcttga

SEQ ID NO: 8 - DNA, 20 bp, artificial sequence
  synthetic
ccacaaagaaacggtgacct

SEQ ID NO: 9 - DNA, 20 bp, artificial sequence
  synthetic
tgctcttcttcgagggtgat

SEQ ID NO: 10 - DNA, 20 bp, artificial sequence
  synthetic
tctcgttgcgtttgtagtgc

SEQ ID NO: 11 - DNA, 20 bp, artificial sequence
  synthetic
catgaacacccagagcattg

SEQ ID NO: 12 - DNA, 20 bp, artificial sequence
  synthetic
atttgtcgtagtggccgttc

SEQ ID NO: 13 - DNA, 20 bp, artificial sequence
  synthetic
ttgctggcactacagaatgc

SEQ ID NO: 14 - DNA, 20 bp, artificial sequence
  synthetic
aacagcctcagagcgacaat

SEQ ID NO: 15 - DNA, 20 bp, artificial sequence
  synthetic
tcgtgtataaggtctgggag

SEQ ID NO: 16 - DNA, 20 bp, artificial sequence
  synthetic
gcagatgtgtccagatgttg

SEQ ID NO: 17 - DNA, 20 bp, artificial sequence
  synthetic
cccaggaagatgaagggttt

SEQ ID NO: 18 - DNA, 20 bp, artificial sequence
  synthetic
ttaagccaatccggtagcac

SEQ ID NO: 19 - DNA, 20 bp, artificial sequence
  synthetic
tgttgttggatatgcccttg

SEQ ID NO: 20 - DNA, 20 bp, artificial sequence
  synthetic
ttgcgctcatcttaggcttt